(19) United States
(12) Patent Application Publication
(10) Pub. No.: US 2021/0299334 A1
(43) Pub. Date: Sep. 30, 2021

(54) Poly(allylguanidine) and the manufacturing process and the use thereof

(71) Applicant: National Taiwan University, Taipei City (TW)

(72) Inventors: TAI-HONG YOUNG, Taipei City (TW); YOU-REN JI, Taipei City (TW)

(21) Appl. No.: 17/060,746

(22) Filed: Oct. 1, 2020

(30) Foreign Application Priority Data
Mar. 27, 2020 (TW) 109110663

(51) Int. Cl. A61L 27/52; C12N 5/0793; C12N 5/00; A61L 27/38

(52) U.S. Cl. CPC A61L 27/52; C12N 5/0619; C12N 2533/30; A61L 27/383; A61L 27/3878; C12N 5/0068

(57) ABSTRACT

The present application provides a poly(allylguanidine) and the manufacturing process thereof. In addition, the present application further provides uses of the poly(allylguanidine), which can be applied in culturing neurons or as an implant for the affected area of a brain tumor after surgical procedure.

### CROSS-REFERENCE TO RELATED APPLICATIONS

This application claims the priority of Republic of China Patent Application No. 109110663 filed on Mar. 27, 2020, in the State Intellectual Property Office of the R.O.C., the disclosure of which is incorporated herein by reference.

### FIELD OF THE INVENTION

The present application relates to a poly(allylguanidine) and the manufacturing process and the use thereof, which can be used as a carrier for neuronal cell culture or an implant of the affected part after brain tumor surgery.

### BACKGROUND OF THE INVENTION

Nerve tissue is mainly composed of neurons and glial cells. Neurons are highly differentiated cells that are structural units and functional units. Mature neurons lack the ability to regenerate (mitosis), and once they are damaged, they cannot regenerate. There are more than ten times of glial cells than neurons. Although glial cells do not transmit nerve impulses, they have the function of supporting and protecting neurons. These cells have the ability to regenerate, even after the damage, they can also be restored.

In the past few decades, many techniques have been used to combine biological materials with biological cells to replace lost or dysfunctional tissues or cells, such as using neurons for nerve repair. The research work of this field is mainly focused on the interaction between cells and their extracellular environment. Recent studies have shown that growth factors or neural stem/precursor cells (NSPC) can support the viability of neurons and the growth of axons. However, NSPC may differentiate into glial cells and gliomas. In fact, when the glial cells form a glial scar after a severe trauma, the neurons cannot regenerate and will lose myelin sheaths in the repair process.

In the neuronal cell culture, it is difficult for the cells to attach to the surface of the culture vessel. In order for the cells to attach well, the hydrophobic surface must be transformed into a more hydrophilic surface while changing the charge on the surface of the culture vessel. The prior art has recognized that the positive charges associated with the surface of biological materials can promote the attachment and growth of neurons. So far, poly-D-lysine (PDL) with structures of butylamine and peptide is the most widely used coating material in the preparation of neuronal cell culture. However, although PDL can promote the viability of neurons, it also enhances the growth of glial cells. In addition, when NSPC is induced on a PDL-coated substrate, a large number of glial cells may be produced.

Accordingly, there is still a need for an ideal biomaterial for neural tissue engineering, which should include improving the viability and functional expression of neurons, and simultaneously preventing or reducing glial cell proliferation and astrocyte hypertrophy, and inducing differentiation of NSPCs into neurons.

### SUMMARY OF THE INVENTION

In view of the problems of the prior art, the present application provides a poly(allylguanidine) comprising the repeating unit represented by formula (1)

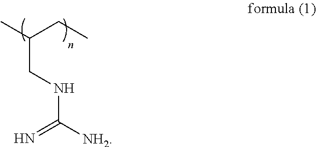

In one embodiment, n is 50-200, the average molecular weight is 4957-19828.

In one embodiment, the poly(allylguanidine) is used as a carrier for neuronal cell culture. Preferably, the poly(allylguanidine) is coated on a cell culture container or a nanofiber sheet for neuronal cell culture in vitro. Preferably, the poly(allylguanidine) is made into a hydrogel or coated on biomedical materials as an implant of the affected part after brain tumor surgery.

In addition, the present application further provides a manufacturing process of poly(allylguanidine), comprising the following steps:
(1) dissolving allylguanidine and 2,2-diamidinyl-2,2-azopropane dihydrochloride (AAPH) in water,
(2) heating the solution of step (1) to 50° C.-80° C., and reacting for 16˜32 hours, and
(3) to dialysis the solution of step (2) to obtain poly(allylguanidine).

In one embodiment, wherein after step (1) further comprises a step of sealing the reaction vessel under argon. Wherein step (2) is heating to 65° C. in an oil bath and reacting for 24 hours.

In one embodiment, the allylguanidine is obtained by the following preparation steps:
(1) adding allylamine to 2-ethyl-thiopseudourea hydrobromide to obtain a mixture;
(2) adding water to the mixture and reacting for 68˜76 hours; and
(3) evaporating the solvent under vacuum to obtain allylguanidine.

In one embodiment, wherein further comprises to recrystallize allylguanidine by ethyl acetate.

The present application provides a poly(allylguanidine) (PAG), which can be used as a polycation coating material for culturing neurons, glial cells, and neural stem/precursor cells (NSPCs). Since allylguanidine (AG) has the guanidino group on the allylic carbon, the synthesized poly(allylguanidine) (PAG) has a guanidino salt. At the same time, PAG has more hydrophobic carbon-carbon backbone structure, so that the high viability of neurons and the low growth ability of glial cells can be simultaneously observed on PAG. This phenomenon is not only affected by the guanidine salt cation of the PAG side chain, but also by the nature of the backbone structure. For biomaterials designed for the application of disease or post-traumatic nerve tissue engineering, poly(allylguanidine) (PAG) provides an ideal carrier material.

The details of the invention are set forth in the following description, which is to be regarded as illustrative methods and materials only, and not restrictive. Other similar or equivalent methods and materials described herein to practice or test the present invention should be regarded as the scope of the resent application. In the specification and the appended claims, the singular form includes the plural as well unless the context clearly indicates otherwise. Unless otherwise defined, all technical and scientific terms used herein have the same meaning as generally understood as one having ordinary skill in the art of the present invention.

### BRIEF DESCRIPTION OF THE DRAWINGS

The application file contains at least one drawing executed in color. Copies of this patent or patent application publication with color drawing(s) will be provided by the Office upon request and payment of the necessary fee. The above and other aspects, features and other advantages of the present invention will be more clearly understood from the following detailed description taken in conjunction with the accompanying drawings, in which:

FIG. 1 shows the immunostaining results of neurons and glial cells after co-cultured on different carriers.

FIG. 2 shows the ratio of neurons and glial cells after co-cultured on different carriers.

FIG. 3 shows the immunostaining results of neurons and glial cells after co-cultured on different carriers.

FIG. 4 shows the immunostaining results of neurons after cultured on different carriers.

FIG. 5 shows the statistical results of the cell viability of neurons after cultured on different carriers.

FIG. 6 shows the statistical results of the neurite length of neurons after cultured on different carriers.

FIG. 7 shows the results of the live/dead cell assay of glial cells after cultured on different carriers.

FIG. 8 shows the statistical results of the cell viability of glial cells after cultured on different carriers.

FIG. 9 shows the immunostaining results of neural stem/precursor cells (NSPCs) after cultured on different carriers.

FIG. 10 shows the ratio of neurons and astrocytes after neural stem/precursor cells (NSPCs) cultured on different carriers.

FIG. 11 shows the results of the live/dead cell assay of GBM8901 after cultured on different concentrations of PAG carriers.

FIG. 12 shows the statistical results of the cell viability of GBM8901 after cultured on different concentrations of PAG carriers. The file of this application contains drawings executed in color.

### DETAILED DESCRIPTION OF THE PREFERRED EMBODIMENT

Reference will be made in detail description to the exemplary embodiments and drawings for being more readily understood to the advantages and features of the present invention, as well as the methods of attaining them. However, the present invention may be carried out in many different forms and should not be construed as limited to the embodiments set forth herein. Conversely, these embodiments are provided to render the present disclosure to be conveyed the scope of the present invention more thoroughly, completely, and fully to one having ordinary skill in the art of the present invention. Moreover, the present invention would be defined only by the appended claims. As used herein, the term “and/or” includes any and all combinations of one or more of the associated listed components.

Unless otherwise defined, all terms (including technical and scientific terms) used herein have the same meaning as generally understood by one having ordinary skill in the art of the present invention. It will be more understandable that, for example, the terms defined in commonly used dictionaries should be understood to have meanings consistent with the contents of the relevant fields, and would not be interpreted overly idealized or overly formal unless clearly defined herein. As described in the present specification, a range of values is used as a shorthand to describe each and every numerical value in the range, and any number within that range may be chosen as the end-value of that range.

The present application provides a poly(allylguanidine) comprising the repeating unit represented by formula (1):

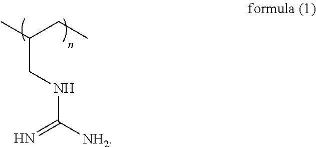

In one embodiment, n is 50-200, the average molecular weight is 4957-19828.

In one embodiment, the poly(allylguanidine) is used as a carrier for neuronal cell culture. Preferably, the poly(allylguanidine) is coated on a cell culture container or a nanofiber sheet for neuronal cell culture in vitro. Preferably, the poly(allylguanidine) is made into a hydrogel or coated on biomedical materials as an implant of the affected part after brain tumor surgery.

In addition, the present application further provides a manufacturing process of poly(allylguanidine), comprising the following steps:
(1) dissolving allylguanidine and 2,2-diamidinyl-2,2-azopropane dihydrochloride (AAPH) in water,
(2) heating the solution of step (1) to 50° C.-80° C., and reacting for 16˜32 hours, and
(3) to dialysis the solution of step (2) to obtain poly(allylguanidine).

In one embodiment, wherein after step (1) further comprises a step of sealing the reaction vessel under argon. Wherein step (2) is heating to 65° C. in an oil bath and reacting for 24 hours.

In one embodiment, the allylguanidine is obtained by the following preparation steps:
(1) adding allylamine to 2-ethyl-thiopseudourea hydrobromide to obtain a mixture;
(2) adding water to the mixture and reacting for 68-76 hours; and
(3) evaporating the solvent under vacuum to obtain allylguanidine.

In one embodiment, wherein further comprises to recrystallize allylguanidine by ethyl acetate.

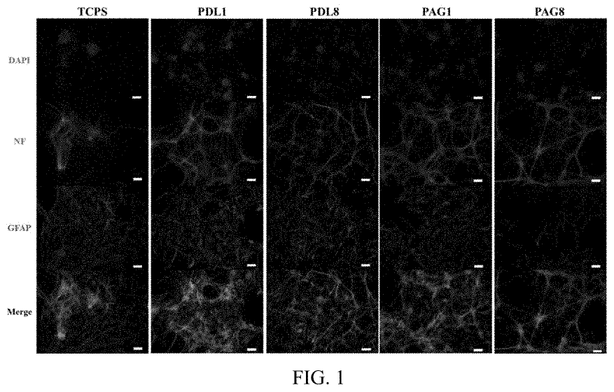

The following examples will show the poly(allylguanidine) provided by the present application, its manufacturing process, as well as the experimental protocols and results as carriers for neuronal cell culture or implants of the affected part after brain tumor surgery. The experiment results are shown in FIG. 1 to FIG. 12. Among them, the experiment results of the examples are merely illustrative and are not intended to limit the scope of the present invention.

### EXAMPLE 1—PREPARATION OF POLY(ALLYLGUANIDINE)

3 g of allylguanidine (AG) and 71 mg of 2,2-diamidinyl-2,2-azopropane dihydrochloride dissolved in 1.058 ml water was poured into a 25 ml round bottom flask that was sealed under argon and placed in an oil bath pre-heated at 65° C., the solution was allowed to react for 24 hours. The product was then added into a dialysis tube in water. After 2 days of dialysis, the product was freeze-dried and a white powder was obtained. The synthesis process is as follows:

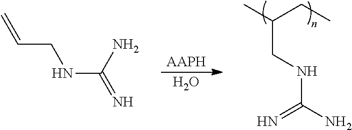

The above-mentioned allylguanidine (AG) can be obtained from commercially available products or by the following preparation process. About 10.07 g of Allylamine was added dropwise to 30.687 g of 2-ethyl-thiopseudourea hydrobromide in a 250 ml flask and stirred for 10 minutes. Subsequently, about 18.5 ml of water was added to the mixture and the resultant solution was stirred at room temperature. After 72 hours, the solvent was evaporated under vacuum at room temperature and the product was recrystallized by ethyl acetate, leading to formation of allylguanidine (AG) as a white crystal, with the yield of 98% (28.833 g). The synthesis process is as follows:

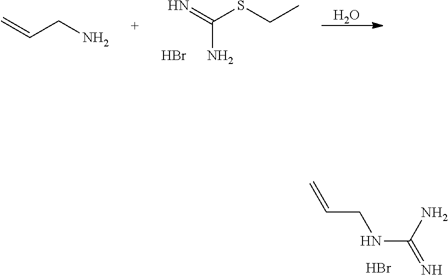

### EXAMPLE 2—PREPARATION OF CARRIERS FOR NEURONAL CELL CULTURE

The poly(allylguanidine) (PAG) is coated on a cell culture container, which can be used for neuronal cell culture in vitro. For example, the poly(allylguanidine) can be coated on a 24-well plate. For the sake of comparison, tissue culture polystyrene (TCPS) was selected as a negative control, and poly-D-lysine (PDL) and/or poly-1-arginine (PLA) were selected as positive controls. Accordingly, PDL and PLA were used to coat onto the internal surface of wells as the positive controls. TCPS represented the uncoated surface of a well as the negative control. poly(allylguanidine) (PAG) was used to coat onto the internal surface of wells as the experimental group. PDL, PLA, and PAG were dissolved in phosphate buffer saline (PBS) at different concentrations of 1 μg/ml and 8 μg/ml, to make dilute and sterilize fluid passed through 0.22 μm micrometers (Millex-GS, US), respectively. 1 ml of such solutions was added into each well of a 24-well plate and incubated (5% CO₂, 37° C.) for one week. Ultimately, the solution was removed and the well was rinsed with PBS before cell seeding.

In addition, poly(allylguanidine) (PAG) can also be coated on nanofiber sheets. For example, poly(acrylonitrile) (PAN) and chemical functionalized poly(acrylonitrile) (f-PAN) nanofiber sheets are selected respectively. Before coating poly(allylguanidine), the nanofibers sheet was soaked in 75 v/v % alcohol for 2 hours and washed twice by PBS. Also, then the coating protocol was the same which PAG coated for 24-well of TCPS.

Furthermore, the poly(allylguanidine) can also be made into a hydrogel or coated on biomedical materials as an implant of the affected part after brain tumor surgery. For example, various weight percentages of allylguanidine/N,N′-methylene bis(acrylamide) were prepared, which aqueous solution contained 1/50 of DMSO or TMZ solution, and contained AAPH and TEMED in the ratio of 0.1 gram and 5.2 microliters per 1 gram of monomer and linker, respectively.

### EXAMPLE 3—CELLS ACQUISITION AND CULTURE

All experiments were conducted according to the guidelines of the National Taiwan University College of Medicine, Laboratory Animal Center. Neurons and glial cells were obtained from 7-day-old Wistar rats' brain and cerebellum. NSPCs were obtained from pregnant Wistar rats' embryos on day 16. 7-day-old Wistar rat was wiped completely with 70% ethanol before decapitation. Under a dissecting microscope, the brain and cerebellum were dissociated. Subsequently, the meninges of the brain and cerebellum were removed and the tissues were dissected in an ice-cold Dulbecco's Modified Eagle's Medium Nutrient Mixture F-12 (Gibco, US) (DMEM/F12, containing 10% fetal bovine serum (FBS), 1% penicillin-streptomycin (PS)) and Krebs-Ringer solution (120 mM NaCl, 25 mM NaHCO₃, 13 mM glucose, 5 mM KCl, 2.5 mM MgSO₄, 1.2 mM KH2PO4, 1% and 0.3% w/v FBS).

For the neuronal cell culture, each cerebellum was minced with scissors and centrifuged at 300 g for 3 min The tissues were resuspended in 5 ml of the Krebs-Ringer solution containing 0.25 w/v trypsin after removal of the supernatant, After adding 200 μl DNase, they were placed in the incubator (5% CO₂, 37° C.) for 30 min. The digestion process was terminated by adding 5 ml of basal modified Eagle's (BME) medium containing 10% FBS, 1% PS, and 25 mM KCl and centrifuging at 300 g for 5 min The sediment was resuspended in the Kerbs-Ringer solution, filtered through a 40 μm mesh, and centrifuged at 300 g for 5 min. The collected pellet was resuspended with the BME medium and seeded into the coated wells at a density of 1×10⁶ cell/well. After 24 hours, 10 μM cytosine arabinoside (Aar-C) was added to the culture medium to stop the proliferation of the glial cells. For the co-culture of the neurons and glial cells, the same procedure was adopted but was lack of the last step of adding Ara-C.

With respect to the glial cells, the brain was minced by a scissor and centrifuged at 300 g for 5 minutes. When the supernatant was removed, the tissue was resuspended in 5 ml of PBS containing 3.42 mM EDTA, 0.25 w/v trypsin, and 200 μl DNase, then placed in an incubator (5% CO₂, 37° C.) for 30 minutes. The digestion process was terminated by adding 5 ml of DMEM/F12 and by centrifuging at 300 g for 5 minutes. The obtained sediment was resuspended in 10 ml DMEM/F12, and seeded into a T75-flask. During a 7 to 8 day time period, the medium was changed after every 3 days and the T75-flask was shaken at 180 g for 30 minutes on an orbital shaker. Subsequently, the supernatant was removed and fresh DMEM/F12 was added. The T75-flask was further shaken at 240 g for 6 hours, followed by removal of the medium, rinsing twice with PBS, adding 10 ml of DMEM/F12 and incubating at 5% CO₂, 37° C. After 5 to 7 days, the medium was removed, the flask was washed twice with PBS, and after adding 2 ml PBS containing 0.05% w/v trypsin, the culture was placed into the incubator (5% CO₂, 37° C.) for 5 minutes. Eventually, 3 ml DMEM/F12 was added to terminate the detaching process of the glial cells, and the culture was centrifuged at 300 g for 5 minutes. The collected pellet was resuspended with DMED/F12 and seeded into the different coated wells at a density of 1×10⁴ cell/well.

Regarding the NSPCs, the rat embryonic cerebral cortices were dissected, cut into small pieces, and collected by centrifugation. The collected pieces were washed with HBSS, crushed, and further resuspended with DMEM/F12 containing N2 supplement (Gibco, US) and 20 ng/mL bFGF (Invitrogen, US). The cerebral cortical NSPCs were cultured in the T25 culture flasks incubated at 37° C. in a humidified atmosphere of 95% air/5% CO₂. In the presence of bFGF, the NSPCs form floating neurospheres. Between the 2nd and 3rd days, the adherent cells were discarded. Also, the neurospheres were collected by centrifugation and sub-cultured in new T25 flasks after mechanical dissociation. The cells grew as new spheres in the following days and became ready for seeding on the 6th day. The obtained neurospheres were centrifuged at 300 g for 5 minutes and resuspended with DMEM/F12 containing N2 supplement, 10% fetal bovine serum (FBS), and 1% penicillin-streptomycin (PS). Eventually, the neurospheres were cultured into the coated wells.

In addition, the glioma cell line GBM8901 was also used in the present invention. The GBM8901 cell line was adding 10 ml of RPMI1640 containing 10% FBS, 1% PS, and incubating at 5% CO₂, 37° C. After 2 to 3 days, the medium was removed, the culture-dish was washed twice with PBS, and after adding 2 ml PBS containing 0.05% w/v trypsin, the culture was placed into the incubator (5% CO₂, 37° C.) for 5 minutes. Eventually, 3 ml RPMI1640 was added to terminate the detaching process of the

GBM8901, and the culture was centrifuged at 300 g for 5 minutes. The collected pellet was resuspended with RPMI1640 and seeded into the different coated wells at a density of 1×10⁴ cell/well.

### EXAMPLE 4—CO-CULTURE

In the present example, the neurons and glial cells obtained in the foregoing example 3 were co-cultured in the 24-well plates of example 2, which includes uncoated TCPS wells, wells coated with 1 μg/mL PDL and PAG (abbreviated PDL1, PAG1), and wells coated with 8 μg/mL PDL and PAG (abbreviated PDL8, PAG8). After 12 days, the immunocytochemical staining was performed.

Please refer to FIG. 1, blue (DAPI) shows the nucleus, green (NF) shows the intermediate filaments of neurons, and red (GFAP) shows glial cells. In terms of neuronal cell culture results (green), both low concentrations of PDL1 and PAG1 and high concentrations of PDL8 and PAG8 have significantly better results compared with the negative control TCPS, but there is no significant difference between PDL and PAG, or between high and low concentrations. However, in terms of the culture results of glial cells (red), the glial cell culture results of low concentrations of PAG1 are slightly less than that of PDL1, and the high concentrations of PAG8 are significantly lesser than the PDL8.

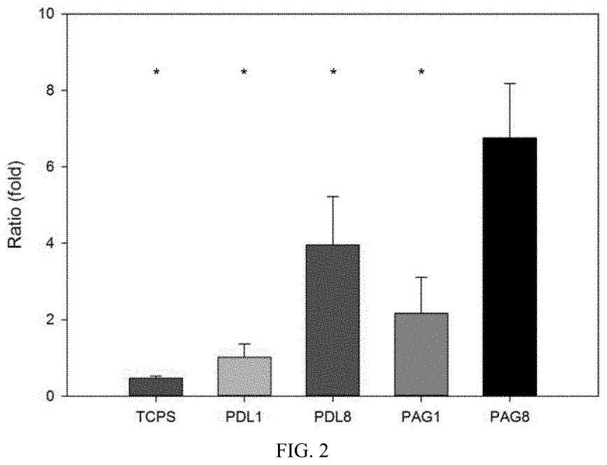

Please refer to FIG. 2, which shows the ratio of neurons and glial cells after cultured. Through the biological image processing and analysis software ImageJ, it can be seen that the ratio of neurons/glial cells of PAG8 is significantly higher than that of all other groups (p<0.05), reaching 6.75±1.42:1.

In addition, the present example further utilizes the poly(acrylonitrile) (PAN) and chemical functionalized poly(acrylonitrile) (f-PAN) nanofibers of the aforementioned example 2. PAG was coated onto the nanofiber sheets, neurons and glial cells were co-cultured for 12 days, then the immunohistochemical staining was performed.

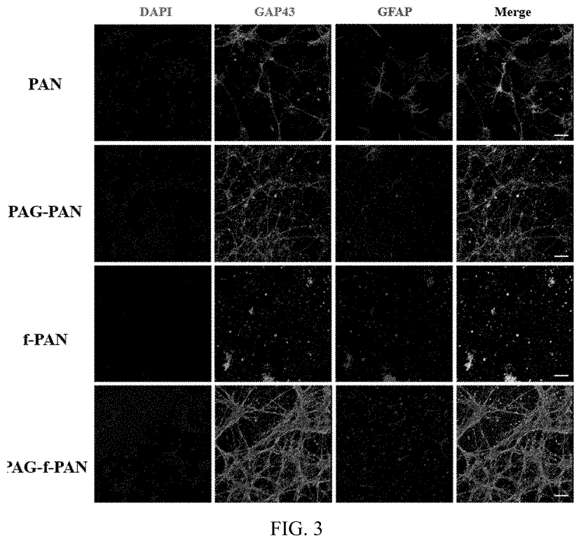

Please refer to FIG. 3, blue (DAPI) shows the nucleus, green (GAP43) shows the neurite, and red (GFAP) shows glial cells. In terms of neuronal cell culture results (green), both PAG-coated PAN and f-PAN nanofiber sheets have significantly better results, especially the PAG-coated f-PAN nanofiber sheets has the best result. In terms of the culture results of glial cells (red), the group coated with PAG also has the result of inhibiting glial cells compared with the control group.

Therefore, it can be known from the present example that PAG with a sufficient coating concentration can support the growth of neurons while limiting the growth of glial cells without the addition of Ara-C to inhibit the proliferation of glial cells. There is an ideal material for the application of nerve tissue engineering, which includes promoting the viability and function of neurons, and preventing or reducing glioma disease.

### EXAMPLE 5—NEURONAL CELL CULTURE

In the present example, the neurons obtained in the foregoing example 3 were cultured in the 24-well plates of example 2, which includes uncoated TCPS wells, wells coated with 1 μg/mL PDL and PAG (abbreviated PDL1, PAG1), and wells coated with 8 μg/mL PDL and PAG (abbreviated PDL8, PAG8). After 12 days, the immunocytochemical staining was performed.

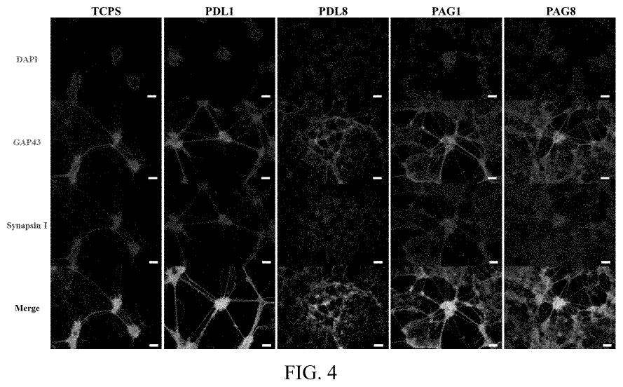

Please refer to FIG. 4, blue (DAPI) shows the nucleus, green (GAP43) shows the neurite, and red (synapsin I) shows synapse. It can be clearly seen from FIG. 4, regardless of the cell number, axons, or synapses, the number of neurons cultured on the PAG-coated plate is significantly more than that of TCPS and PDL, and the high concentration of PAG8 is better than the low concentration of PAG1.

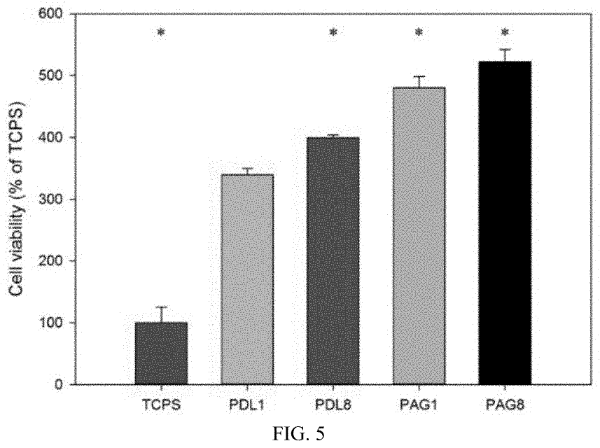

Please refer to FIG. 5, which shows the statistical results of the cell viability. Through the Alamar Blue assay (Thermo-Fisher, US), it can be seen that the cell viability of neurons cultured on the PAG-coated plate is significantly higher than the groups of TCPS and PDL, and the high concentration of PAG8 is better than the low concentration of PAG1.

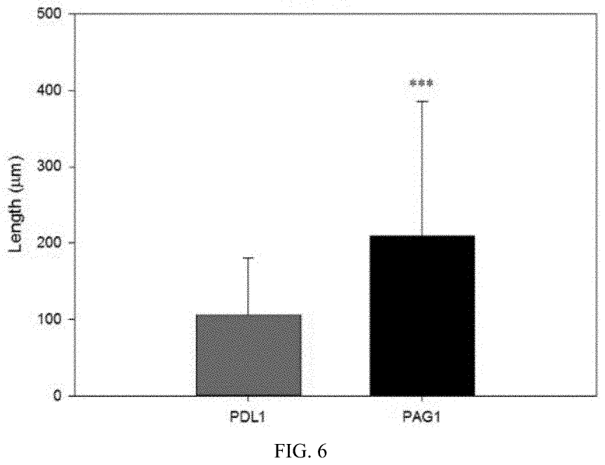

Please refer to FIG. 6, which shows the statistical results of the neurite length. The calculation results of the biological image processing and analysis software ImageJ shows that neurons cultured on the PAG-coated culture plate has better neurite length than the PDL group.

PDL is the current standard material for culturing neurons, while the results of the present example indicate that PAG could maintain a suitable environment for supporting an extended neural network with axonal growth and synaptogenesis, even exhibited higher neuronal viability than PDL.

### EXAMPLE 6—GLIAL CELL CULTURE

In the present example, the glial cells obtained in the foregoing example 3 were cultured in the 24-well plates of example 2, which includes uncoated TCPS wells, wells coated with 1 μg/mL PDL and PAG (abbreviated PDL1, PAG1), and wells coated with 8 μg/mL PDL and PAG (abbreviated PDL8, PAG8). After 7 days, assays were performed. Near-pure glial cells were cultured by a lack of using Ara-C.

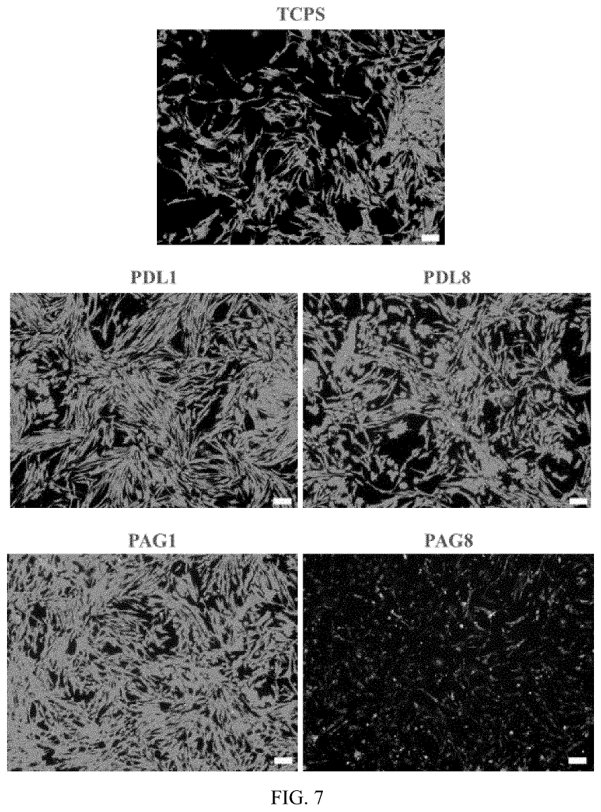

Please refer to FIG. 7, which shows the results of the live/dead cell assay of glial cells. As can be seen, glial cells could continuously increase viability on PDL, and the group of low PAG concentration (PDL 1) also had the same result. However, the obvious inhibition of glial cell viability was found on the culture plate coated with high PAG concentration (PDL8), only a few glial cells survive on PDL8.

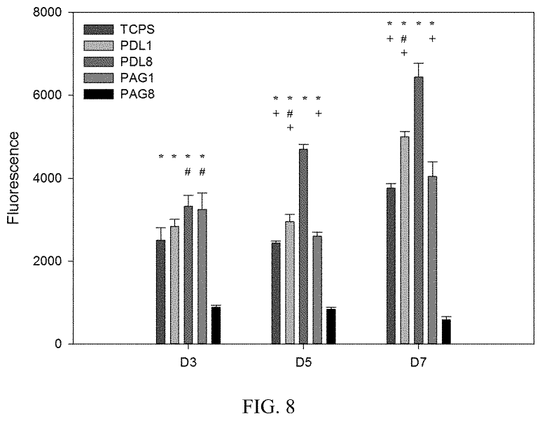

Please refer to FIG. 8, which shows the statistical results of the cell viability of glial cells. The cell viability of glial cells was tested on the 3^{rd}, 5^{th}, and 7^{th} days after cultured. As can be seen, glial cells could continuously increase viability on PDL from day 3 to day 7. The same results were obtained in the low PAG concentration group (PDL1), while the obvious inhibition of glial cell viability was found on the culture plate coated with high PAG concentration (PDL8).

Therefore, the results of the present example show that high concentrations of PAG (PDL8) as a carrier can significantly inhibit the growth and viability of glial cells, and this effect cannot be achieved by PDL materials of prior art.

### EXAMPLE 7—NSPC CULTURE

In the present example, the neurons obtained in the foregoing example 3 were cultured in the 24-well plates of example 2, which includes uncoated TCPS wells, and wells coated with 8 μg/mL PDL, PLA and PAG (abbreviated PDL8, PLA8, and PAG8). After 7 days, the immunocytochemical staining was performed.

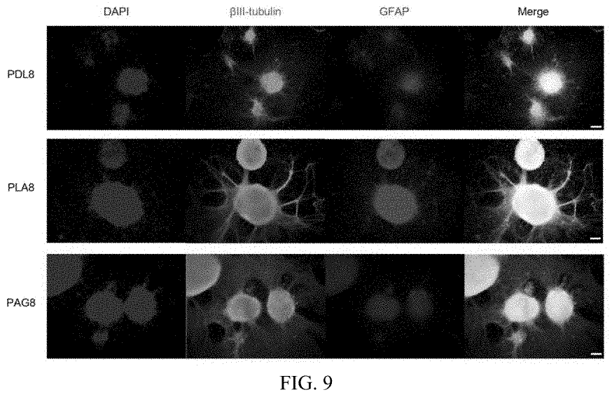

Please refer to FIG. 9, blue (DAPI) shows the nucleus, green (βIII-tubulin) shows the tubulin of neurons, and red (GFAP) shows glial cells. It can be clearly seen from FIG. 9, many NSPCs cultured on PLA8 and PAG8 differentiated into neurons (green). Comparing with the NSPCs cultured on PDL, which number was less than PLA8 and PAG8 even if there were also differentiated into neurons. Most of the NSPCs cultured on PDL8, PLA8, and PAG8 also differentiated into astrocytes. Compared with the number of differentiated into neurons, however, only the NSPCs cultured on PAG8 found that the number of differentiated astrocytes is significantly less than that of neurons. The difference in the number of astrocytes and neurons on PDL8 and PLA8 is not significant.

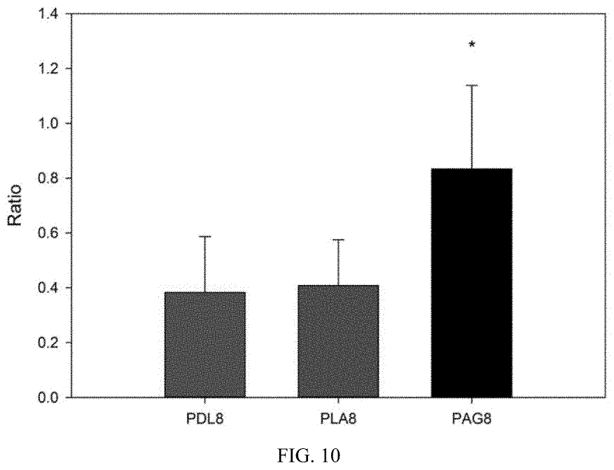

Please refer to FIG. 10, which shows the statistical results of the neurons and astrocytes ratio. It is more obvious from the statistical chart that the ratio of neurons and astrocytes cultured on PAG8 is significantly higher than that of PDL8 and PLA8, indicating that PAG has the effect of promoting the growth of neurons and inhibiting the growth of astrocytes.

Accordingly, PAG hydrogel or PAG-coated biomaterials are potential materials in nerve treatment via regulation of micro-environment to support neuron outgrowth, to prevent gliosis and astrocytic hypertrophy, and promote differentiation of NSPCs to neurons.

### EXAMPLE 8—GLIOMA CELL LINE GBM8901 CULTURE

In the present example, the glioma cell line GBM8901 treated in the foregoing example 3 was cultured in the 24-well plates of example 2, which includes uncoated TCPS wells, and wells coated with PGA of 1 μg/mL, 4 μg/mL, and 8 μg/mL (abbreviated PAG1, PAG4, and PAG8). After 3 days, the immunocytochemical staining was performed.

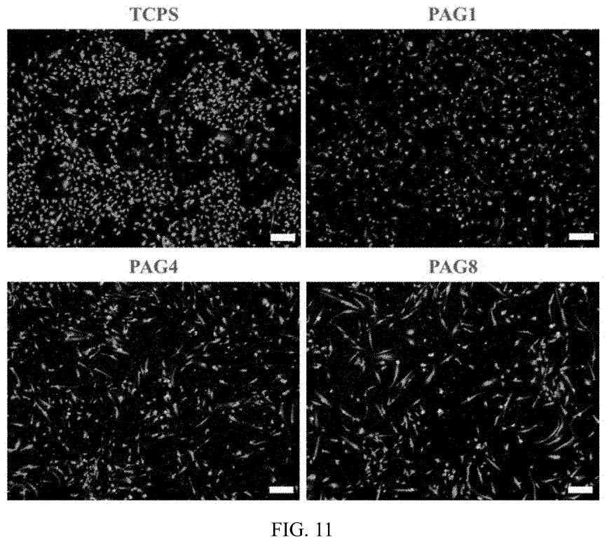

Please refer to FIG. 11, which shows the results of the live/dead cell assay of GBM8901. It can be seen that GBM8901 survives well when cultured on TCBS. In contrast, there are fewer GBM8901 cells that survived in the wells coated with various concentrations of PAG (PAG1, PAG4 and PAGE) after cultured. It is obvious that PAG can inhibit the viability of glioma cells.

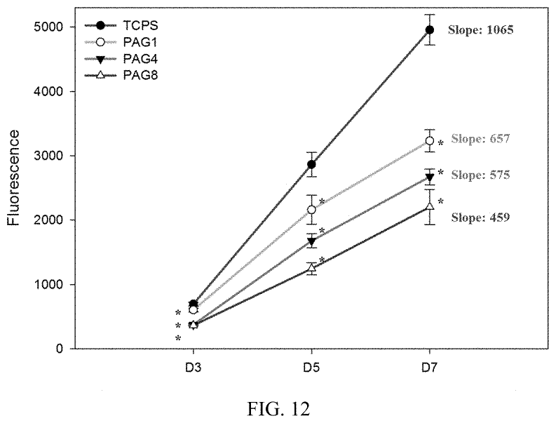

Please refer to FIG. 12, which shows the statistical results of the cell viability of GBM8901. The viability of GBM8901 was tested on the 3^{rd}, 5^{th}, and 7^{th} days after cultured. As can be seen, GBM8901 could continuously increase viability on TCBS and PAG from day 3 to day 7, but the increased degree of GBM8901 viability on PAG is significantly less than that of TCBS. Moreover, the increased degree of GBM8901 viability is lesser and lesser as the concentration of PAG increases, indicating that PAG can indeed reduce the proliferation of glioma cells.

Therefore, the results of the present example show that PAG as a carrier can significantly reduce the growth and viability of glioma cells, and the higher the concentration of PAG used, the better the effect achieved. Obviously, PAG can be used as an ideal material for implants in affected parts after brain tumor surgery.

What is claimed is:

1. A poly(allylguanidine) comprising the repeating unit represented by formula (1):

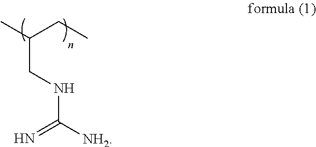

2. The poly(allylguanidine) of claim 1, wherein n is 50-200, the average molecular weight is 4957-19828.

3. A use of the poly(allylguanidine) of claim 1, comprising the use of the poly(allylguanidine) as a carrier for neuronal cell culture.

4. The use of claim 3, wherein the poly(allylguanidine) is coated on a cell culture container or a nanofiber sheet for neuronal cell culture in vitro.

5. The use of claim 3, wherein the poly(allylguanidine) is made into a hydrogel or coated on biomedical materials as an implant of the affected part after brain tumor surgery.

6. A manufacturing process of poly(allylguanidine), comprising the following steps:
(1) dissolving allylguanidine and 2,2-diamidinyl-2,2-azopropane dihydrochloride in water,
(2) heating the solution of step (1) to 50° C.-80° C., and reacting for 16-32 hours, and
(3) to dialysis the solution of step (2) to obtain poly(allylguanidine).

7. The manufacturing process of claim 6, wherein after step (1) further comprises a step of sealing the reaction vessel under argon.

8. The manufacturing process of claim 6, wherein step (2) is heating to 65° C. in an oil bath and reacting for 24 hours.

9. The manufacturing process of claim 6, wherein the allylguanidine is obtained by the following preparation steps:
(4) adding allylamine to 2-ethyl-thiopseudourea hydrobromide to obtain a mixture;
(5) adding water to the mixture and reacting for 68-76 hours; and
(6) evaporating the solvent under vacuum to obtain allylguanidine.

10. The manufacturing process of claim 9, wherein further comprises to recrystallize allylguanidine by ethyl acetate.
